(19) United States
(12) Patent Application Publication
(10) Pub. No.: US 2025/0020655 A1
(43) Pub. Date: Jan. 16, 2025

(54) METHOD FOR ANALYZING LEUCINE AND ISOLEUCINE, AS WELL AS DEVICE FOR ANALYZING LEUCINE AND ISOLEUCINE

(71) Applicants: SHIMADZU CORPORATION, Kyoto (JP); NATIONAL UNIVERSITY CORPORATION SHIMANE UNIVERSITY, Shimane (JP)

(72) Inventors: Takanari HATTORI, Kyoto-shi (JP); Jun WATANABE, Kyoto-shi (JP); Hironori KOBAYASHI, Izumo-shi (JP); Yoshitomo NOTSU, Izumo-shi (JP)

(73) Assignees: SHIMADZU CORPORATION, Kyoto (JP); NATIONAL UNIVERSITY CORPORATION SHIMANE UNIVERSITY, Shimane (JP)

(21) Appl. No.: 18/760,543

(22) Filed: Jul. 1, 2024

(30) Foreign Application Priority Data
Jul. 12, 2023 (JP) 2023-114704

(51) Int. Cl. G01N 33/68

(52) U.S. Cl. CPC G01N 33/6812; G01N 33/6848

(57) ABSTRACT

A method for analyzing leucine and isoleucine for newborn mass-screening or similar tests by multiple reaction monitoring (MRM) measurements using a tandem mass spectrometer is provided. The method includes: a measurement step for acquiring intensity information of ions originating from leucine and isoleucine by performing, on a specimen, a first MRM measurement for at least one MRM transition selected from a group of m/z 132>69, m/z 132>57, m/z 132>56, m/z 132>43, m/z 132>41, m/z 132>39, m/z 132>30 and m/z 132>27 as well as a second MRM measurement for an MRM transition of m/z 132>86; and a processing step for discriminating whether the specimen contains leucine or isoleucine, or for determining a relative abundance of leucine and isoleucine in the specimen, based on a qualifier ion ratio which is a ratio between a signal intensity obtained by the first MRM measurement and a signal intensity obtained by the second MRM measurement.

### TECHNICAL FIELD

The present invention relates to a method and device for analyzing leucine and isoleucine.

### BACKGROUND ART

Newborn screening tests have been widely carried out in order to detect and treat inborn errors of metabolism or similar abnormalities of newborns in their early phases. In recent years, with the rapid progress of the technique of mass spectrometry, newborn mass-screening tests which use tandem mass spectrometers have also been popularly conducted, exhibiting significant effects on the early detection of inborn errors of metabolism of newborns (see Non Patent Literature 1).

One of the aforementioned types of newborn mass-screening tests is an analysis of leucine and isoleucine for diagnosing maple syrup urine disease. In a normal analysis of leucine and isoleucine, a multiple reaction monitoring (MRM) measurement using a tandem mass spectrometer is performed for a quantitative analysis in which a precursor ion originating from leucine and isoleucine is combined with a characteristic product ion of m/z 132>86.

### CITATION LIST

### Non Patent Literature

Non Patent Literature 1: Yousuke Shigematsu, “The Realities of Newborn Screening by Tandem Mass Spectrometry in Japan”, Journal of the Mass Spectrometry Society of Japan, Vol. 64, No. 4, 2016, pp. 127-131

### SUMMARY OF INVENTION

### Technical Problem

In a newborn mass-screening test, a huge number of specimens (“analyte samples”) must be quickly analyzed. Therefore, a flow injection analysis (FIA) method has conventionally been used for the measurement, in which the samples set in an autosampler are sequentially introduced into a mass spectrometer along with a mobile phase without being made to flow through a column in a liquid chromatograph (LC). In the flow injection analysis method, since each sample is introduced into the mass spectrometer without undergoing component separation by the column, various substances contained in the sample are simultaneously subjected to the MRM measurement.

However, since leucine and isoleucine are structural isomers and identical in mass number, it is impossible to separate the two compounds from each other for quantitative determination when the FIA method is used for the MRM measurement. To address this problem, maple syrup urine disease has conventionally been diagnosed in two stages, including the primary mass-screening test which uses an MRM measurement employing the FIA method and the secondary mass-screening test which uses an “on-column analysis”, a type of mass spectrometry which includes component separation by a column. Specifically, in the primary test, the total of the quantitative values of leucine and isoleucine is compared with a predetermined cutoff value. If that total is lower than the cutoff value, the sample is judged to be negative (i.e., not positive for maple syrup urine disease), whereas the secondary test is performed when the total is not lower than the cutoff value.

In the secondary test, leucine, isoleucine and allo-isoleucine (which is an optical isomer of isoleucine) are separated from each other and individually subjected to a quantitative analysis, and the quantitative value of leucine is compared with a previously set cutoff value. If the quantitative value of leucine is lower than the cutoff value, the sample is judged to be negative. If that value is not lower than the cutoff value, whether allo-isoleucine has or has not been detected is additionally determined. If allo-isoleucine has been detected, the sample is judged to be “detailed examination required”. If allo-isoleucine has not been detected, the sample is judged to be “retesting required”.

Thus, the diagnosis of maple syrup urine disease requires two-stage mass-screening tests. Since the newborn screening test normally uses an analyte sample prepared from blood obtained from a newborn, performing the secondary test will physically impose a heavy burden on the newborn. Performing the secondary test will also put a heavy psychological burden on the parents of the newborn who has not been tested negative in the primary test. Furthermore, since it is often the case that the primary and secondary tests are carried out by different facilities, additional time and labor may be required for sending the specimen to the second facility which carries out the secondary test. Therefore, it is desirable to perform separate detection of leucine and isoleucine in the stage of the primary test in order to reduce the number of specimens to be transferred to the secondary test.

The problem to be solved by the present invention is to provide an analyzing method capable of discriminating between leucine and isoleucine in an MRM measurement for newborn mass-screening or similar purposes.

### Solution to Problem

A method for analyzing leucine and isoleucine according to the present invention developed for solving the previously described problem is a method for analyzing leucine and isoleucine using a tandem mass spectrometer, the method including:
a measurement step for acquiring intensity information of ions originating from leucine and isoleucine by performing, on a specimen, a first MRM measurement for at least one multiple reaction monitoring (MRM) transition selected from the group of m/z 132>69, m/z 132>57, m/z 132>56, m/z 132>43, m/z 132>41, m/z 132>39, m/z 132>30 and m/z 132>27 as well as a second MRM measurement for an MRM transition of m/z 132>86; and
a processing step for discriminating whether the specimen contains leucine or isoleucine, or for determining a relative abundance of leucine and isoleucine in the specimen, based on a qualifier ion ratio which is a ratio between a signal intensity obtained by the first MRM measurement and a signal intensity obtained by the second MRM measurement.

A device for analyzing leucine and isoleucine according to the present invention developed for solving the previously described problem includes:
a measurement unit which is a tandem mass spectrometer configured to perform, on a specimen, a first MRM measurement for at least one multiple reaction monitoring (MRM) transition selected from the group of m/z 132>69, m/z 132>57, m/z 132>56, m/z 132>43, m/z 132>41, m/z 132>39, m/z 132>30 and m/z 132>27 as well as a second MRM measurement for an MRM transition of m/z 132>86; and
a processing unit configured to discriminate whether the specimen contains leucine or isoleucine, or to determine a relative abundance of leucine and isoleucine in the specimen, based on a qualifier ion ratio which is a ratio between a signal intensity obtained by the first MRM measurement and a signal intensity obtained by the second MRM measurement in the measurement unit.

### Advantageous Effects of Invention

In the method for analyzing leucine and isoleucine as well as the device for analyzing leucine and isoleucine according to the present invention, two MRM transitions are used for discriminating between leucine and isoleucine or determining the relative abundance of leucine and isoleucine: the MRM transition of a target ion (m/z 132>86) which shows almost the same ion intensity for the same amount of target substance regardless of whether the target substance actually contained in the specimen is leucine or isoleucine; and the MRM transition of a qualifier ion (m/z 132>69, m/z 132>57, m/z 132>56, m/z 132>43, m/z 132>41, m/z 132>39, m/z 132>30 or m/z 132>27) which shows a different ion intensity for the same amount of target substance depending on whether the target substance actually contained in the specimen is leucine or isoleucine. Therefore, it is possible to discriminate between leucine and isoleucine without separating the two compounds by means of a column of a liquid chromatograph.

### BRIEF DESCRIPTION OF DRAWINGS

FIG. 1 is a configuration diagram of the main components of one embodiment of a liquid chromatograph mass spectrometer according to the present invention.

FIG. 2 is a chromatogram waveform diagram acquired at an MRM transition of m/z 132.10>86.10 and an MRM transition of m/z 132.10>69.20 for a sample containing only isoleucine (Leu:Ile=0:100).

FIG. 3 is a chromatogram waveform diagram acquired at an MRM transition of m/z 132.10>86.10 and an MRM transition of m/z 132.10>69.20 for a sample containing only leucine (Leu:Ile=100:0).

FIGS. 4A-4H are graphs showing how the qualifier ion ratios for leucine and isoleucine depend on the collision energy when an MRM transition of m/z 132.10>86.10 is designated as the target ion and each of the following MRM transitions is designated as the qualifier ion: m/z 132.10>69.20, m/z 132.10>57.10, m/z 132.10>56.10, m/z 132.10>43.10, m/z 132.10>41.10, m/z 132.10>39.10, m/z 132.10>30.20, m/z 132.10>27.20.

FIGS. 5A-5H are diagrams showing the relationship between the percentage of leucine in the sample and the qualifier ion ratio when an MRM transition of m/z 132.10>86.10 is designated as the target ion and each of the following MRM transitions is designated as the qualifier ion: m/z 132.10>69.20, m/z 132.10>57.10, m/z 132.10>56.10, m/z 132.10>43.10, m/z 132.10>41.10, m/z 132.10>39.10, m/z 132.10>30.20, m/z 132.10>27.20.

FIG. 6 is a table showing analysis conditions for MRM measurements.

### DESCRIPTION OF EMBODIMENTS

One embodiment of the method for analyzing leucine and isoleucine according to the present invention is hereinafter described with reference to the drawings. For example, the method for analyzing leucine and isoleucine according to the present embodiment may be used in a newborn mass-screening test for diagnosing maple syrup urine disease.

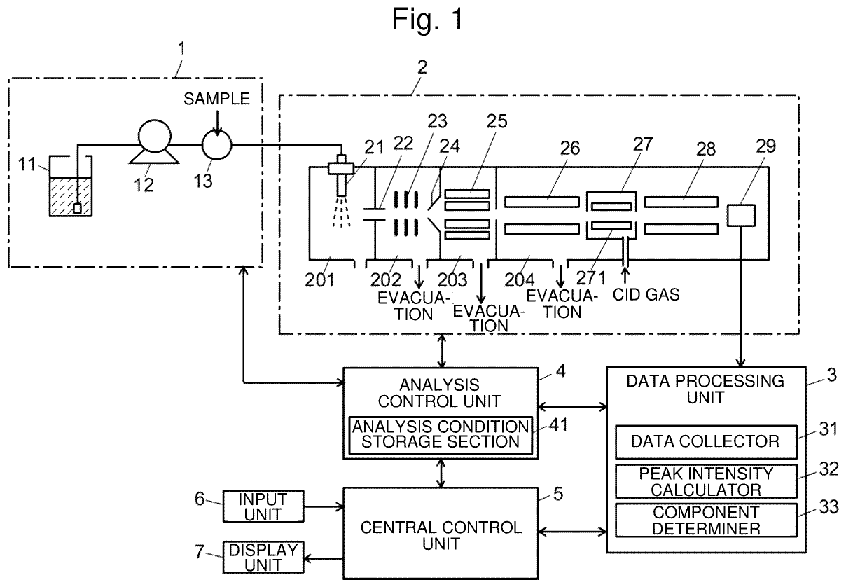

FIG. 1 is a configuration diagram of the main components of a liquid chromatograph mass spectrometer used in the present embodiment. The liquid chromatograph mass spectrometer in the present embodiment includes a liquid chromatograph 1, mass analyzer 2, data processing unit 3, analysis control unit 4, central control unit 5, input unit 6 and display unit 7.

The liquid chromatograph 1 includes: a mobile phase container 11 in which a mobile phase is stored; a pump 12 for drawing the mobile phase (solvent) and supplying it at a constant flow rate; an injector 13 for injecting a liquid sample into the mobile phase; and an autosampler (not shown) connected to the injector 13. Although the liquid chromatograph 1 has a column for separating components in a liquid sample, this column is not used in the present embodiment; the liquid sample is introduced into the mass analyzer 2 along with the mobile phase by a flow injection analysis (FIA) method. In a normal newborn mass-screening test, the sample is prepared from blood extracted from a piece of filter paper on which the blood has been placed, as described in Non Patent Literature 1. However, the sample is not limited to this form; it may also be prepared from urine or other kinds of body fluids.

The mass analyzer 2 is a triple quadrupole mass spectrometer which is a type of tandem mass spectrometer. It includes an ionization chamber 201 maintained at substantially atmospheric pressure, as well as a first intermediate vacuum chamber 202, second intermediate vacuum chamber 203 and high vacuum chamber 204 individually evacuated by means of vacuum pumps (not shown). The ionization chamber 201 is provided with an ESI spray 21 configured to perform ionization by electrospray ionization (ESI). The ionization chamber 201 is connected to the first intermediate vacuum chamber 202 in the next stage by a desolvation tube 22. The first intermediate vacuum chamber 202 contains an ion guide 23 configured to transport ions while converging them. The first intermediate vacuum chamber 202 communicates with the second intermediate vacuum chamber 203 in the next stage through a small hole formed at the apex of a skimmer 24. The second intermediate vacuum chamber 203 also contains a multipole ion guide 25 configured to transport ions while converging them.

Within the high vacuum chamber 204, a front quadrupole mass filter 26, collision cell 27, rear quadrupole mass filter 28 and ion detector 29 are arranged along the direction of the flow of ions. The collision cell 27 contains a quadrupole ion guide 271. Each of the front and rear quadrupole mass filters 26 and 28 has the function of selectively allowing an ion having a predetermined mass-to-charge ratio to pass through. The collision cell 27 is configured to receive inert collision-induced dissociation (CID) gas, such as argon gas, introduced from an external source to give this cell the function of fragmenting an introduced ion into product ions by causing the ion to come in contact with the CID gas.

The data processing unit 3 is configured to receive detection data from the ion detector 29 and perform processing operations based on those data. This unit includes a data collector 31, peak intensity calculator 32 and component determiner 33 as its functional blocks. The analysis control unit 4 is configured to control the operations of the liquid chromatograph 1 and the measurement unit 2 according to an analysis condition file stored in an analysis condition storage section 41. The central control unit 5 is mainly configured to perform an overall control as well as a control of the user interface through the input unit 6, display unit 7 and other devices.

In general, the data processing unit 3, analysis control unit 4 and central control unit 5 are actually a personal computer or a more sophisticated type of computer called a “workstation”, on which the functions of the aforementioned functional blocks are realized by executing, on the computer, dedicated software (computer program) preinstalled on that same computer.

### [Outline of MRM Measurement Operation]

In the liquid chromatograph mass spectrometer shown in FIG. 1, MRM measurements for predetermined MRM transitions are repeatedly performed with the mass spectrometer 2 in an analysis of leucine and isoleucine. The operation during those MRM measurements is hereinafter outlined.

In the liquid chromatograph 1, the liquid supply pump 12 draws a mobile phase from the mobile phase container 11 and sends it to the injector 13 at a substantially constant flow velocity. The injector 13 puts a predetermined amount of sample (specimen) into the mobile phase at a predetermined timing. The sample is carried by the flow of the mobile phase and arrives at the ESI spray 21 in the measurement unit 2. While travelling through the passage leading to the ESI spray 21, the sample is diffused in the travelling direction. Therefore, the amount of sample introduced into the ESI spray 21 is initially at an extremely low level and then rapidly increases. After reaching its peak point, the amount rapidly decreases to zero. That is to say, the concentration distribution of the sample with respect to time forms a peak shape which is approximate to a Gaussian distribution.

At the ESI spray 21, the sample is transformed into fine electrically charged droplets and sprayed into the ionization chamber 201. Those charged droplets come in contact with the residual gas molecules and are split into even finer droplets, with the solvent in the droplets turning into vapor. During this process, the compound molecules in the sample are ionized. The resulting ions are transferred through the desolvation tube 22 into the first intermediate vacuum chamber 202, from which they are further transferred through the ion guide 23, small hole of the skimmer 24, and multipole ion guide 25 to the high vacuum chamber 204. The ions originating from the sample are introduced into the front quadrupole mass filter 26, where only an ion having a predetermined mass-to-charge ratio corresponding to the voltages applied to the electrodes forming the front quadrupole mass filter 26 is selectively allowed to pass through as a precursor ion. The precursor ion enters the collision cell 27 and undergoes dissociation by coming in contact with the CID gas, whereby various productions are generated.

The various product ions thus generated are introduced into the rear quadrupole mass filter 28, where only a product ion having a predetermined mass-to-charge ratio corresponding to the voltages applied to the electrodes forming the rear quadrupole mass filter 28 is selectively allowed to pass through and reach the ion detector 29. The ion detector 29 produces a detection signal corresponding to the amount of ions it has received. This signal is converted into digitized detection data by an analogue-to-digital converter (not shown) and sent to the data processing unit 3.

The analysis control unit 4 operates the mass analyzer 2 so that appropriate voltages for the intended MRM transition are applied to the electrodes in the front and rear quadrupole mass filters 26 and 28. This operation yields detection data which represents the ion intensity of an ion corresponding to a specific MRM transition among the ions originating from a compound contained in the sample, i.e., a product ion having a specific mass-to-charge ratio resulting from the dissociation of a precursor ion having a specific mass-to-charge ratio.

### [Principle of Method for Analyzing Leucine and Isoleucine]

Next, the principle of the characteristic method for analyzing leucine and isoleucine according to the present embodiment is described.

In the mass analyzer 2, the precursor ion undergoes dissociation through the CID process within the collision cell 27. The form of dissociation varies depending on the amount of kinetic energy possessed by the precursor ion. This amount of kinetic energy is the collision energy (CE). The collision energy is determined by the DC potential difference between the entrance end of the collision cell 27 and an element located immediately before the cell 27 (in FIG. 1, this element is the front quadrupole mass filter 26, although it may be a different type of ion optical element, such as an additional ion lens). Therefore, the collision energy is normally indicated by that potential difference. Accordingly, the collision energy can be regulated by a DC bias voltage or voltages applied to one or both of the front quadrupole mass filter 26 and the ion optical element located before this mass filter 26.

As noted earlier, changing the collision energy causes a change in the form of dissociation of the precursor ion, so that the generation pattern of the plurality of kinds of product ions also changes. In order to improve the detection sensitivity, a product ion (i.e., an MRM transition) having the highest possible level of ion intensity should preferably be selected. Therefore, m/z 132.10>86.10, which yields the highest ion intensity, has been used as the MRM transition in the conventional analysis of leucine and isoleucine. However, as noted earlier, it is impossible to discriminate between leucine and isoleucine when m/z 132.10>86.10 is used. To address this problem, the present inventors conducted a detailed experimental study of the relationship between collision energy and ion intensity for various MRM transitions related to leucine and isoleucine.

The experiment was performed according to the following procedure:

A 100-μg/L aqueous solution of leucine and that of isoleucine were prepared from reference standards of the two compounds, respectively, using ultrapure water. Using each solution as a sample, a product-ion scan measurement for a precursor ion (m/z 132.10) common to leucine and isoleucine was initially performed, using a liquid chromatograph mass spectrometer manufactured by SHIMADZU CORPORATION, model number LCMS-8060, to search for a product ion specific to the compound in question. The result demonstrated that no specific product ion originating from only one of those compounds was observed, while there were nine representative mass-to-charge ratios at which product ions can be detected with certain levels of peak intensity: m/z 86.10, m/z 69.20, m/z 57.10, m/z 56.10, m/z 43.10, m/z 41.10, m/z 39.10, m/z 30.20 and m/z 27.20.

For each of the nine product ions, the peak intensity was measured for various setting values of the collision energy ranging from −100 V to −5 V in steps of 5 V. The result showed that there was no practical difference between leucine and isoleucine in terms of the peak-intensity pattern with respect to the change in collision energy in the case of m/z 132.10>86.10 which is the MRM transition conventionally used for the quantitative determination of leucine and isoleucine. On the other hand, a significant difference in the peak-intensity pattern was observed between leucine and isoleucine in the cases of the eight remaining MRM transitions (m/z 132.10>69.20, m/z 132.10>57.10, m/z 132.10>56.10, m/z 132.10>43.10, m/z 132.10>41.10, m/z 132.10>39.10, m/z 132.10>30.20 and m/z 132.10>27.20).

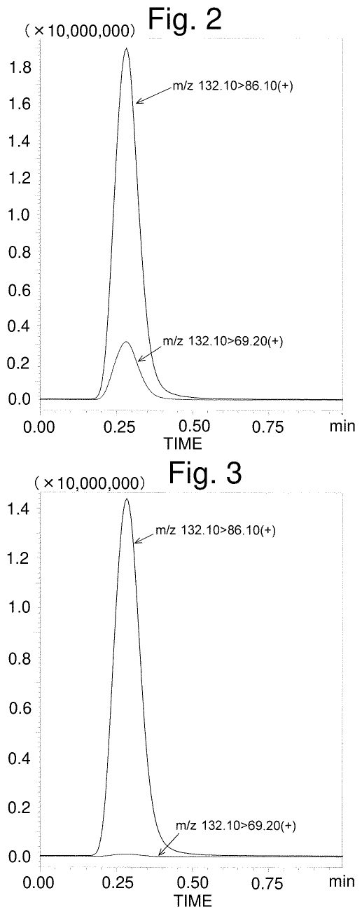

For example, FIGS. 2 and 3 are chromatogram waveform diagrams respectively acquired for a sample containing only isoleucine (Leu:Ile=0:100) and a sample containing only leucine (Leu:Ile=100:0) at an MRM transition of m/z 132.10>86.10 and an MRM transition of m/z 132.10>69.20. In both cases, the component concentration was 100 μg/L. As can be seen in FIGS. 2 and 3, the leucine-containing sample and the isoleucine-containing sample had similar peak-intensity patterns at m/z 132.10>86.10, whereas they had significantly different peak-intensity patterns at m/z 132.10>69.20. Furthermore, the intensity of the peak at m/z 132.10>69.20 was lower than that of the peak at m/z 132.10>86.10 in both of the leucine-containing and isoleucine-containing samples. These experimental findings demonstrate that it is possible to discriminate between leucine and isoleucine by making use of the difference between the peak-intensity pattern at first MRM transition of m/z 132.10>86.10 and one or more of the peak-intensity patterns at the eight other MRM transitions (m/z 132.10>69.20, m/z 132.10>57.10, m/z 132.10>56.10, m/z 132.10>43.10, m/z 132.10>41.10, m/z 132.10>39.10, m/z 132.10>30.20 and m/z 132.10>27.20).

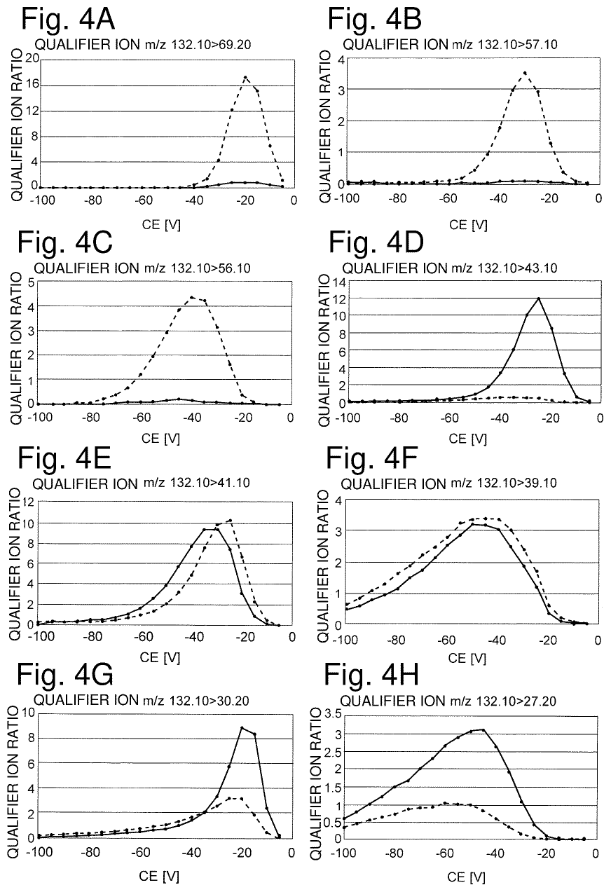

Accordingly, a signal intensity A1 of a peak at the MRM transition of m/z 132.10>86.10, with the collision energy set at −10 V, and a signal intensity A2 of a peak at each of the eight aforementioned MRM transitions, with the setting value of the collision energy varied within a range of −100 V to −5 V in steps of 5 V, were measured, and the ratio of the measured values (A2/A1) multiplied by 100 was calculated. FIGS. 4A-4H show the measured results.

The ion detected at the MRM transition of m/z 132.10>86.10 can be considered as a target ion, while each of the ions detected at the eight other MRM transitions (m/z 132.10>69.20, m/z 132.10>57.10, m/z 132.10>56.10, m/z 132.10>43.10, m/z 132.10>41.10, m/z 132.10>39.10, m/z 132.10>30.20 and m/z 132.10>27.20) can be considered as a qualifier ion. In that case, the intensity ratio of the two ions corresponds to the so-called “qualifier ion ratio”. The aforementioned value, (A2/A1)×100, is hereinafter called the qualifier ion ratio.

FIG. 4A-4H are graphs showing the collision-energy dependency of the qualifier ion ratio at the eight MRM transitions for leucine and isoleucine. In those graphs, the horizontal axis represents the collision energy, while the vertical axis represents the qualifier ion ratio (A2/A1×100). The solid line in those graphs indicates the qualifier ion ratio for leucine, while the broken line indicates the qualifier ion ratio for isoleucine. As can be seen in those graphs, a difference in the pattern of the collision-energy dependency of the qualifier ion ratio was observed between leucine and isoleucine at any of the eight MRM transitions. In particular, the qualifier ion ratio at m/z 132.10>39.10 showed a clear difference over the almost entire range of collision energy (FIG. 4F). The qualifier ion ratio at m/z 132.10>41.10 also showed a clear difference depending on the value of the collision energy (FIG. 4E). At the other MRM transitions, i.e., m/z 132.10>69.20, m/z 132.10>57.10, m/z 132.10>56.10, m/z 132.10>43.10, m/z 132.10>30.20 and m/z 132.10>27.20, a remarkable difference in the qualifier ion ratio was observed depending on the value of the collision energy (FIGS. 4A-4D, 4G and 4H). For example, both of the qualifier ion ratios of leucine and isoleucine at m/z 132.10>69.20 had peaks (local maxima) at −20 V of collision energy, and the difference in the qualifier ion ratio between the two compounds was the largest at those peaks (FIG. 4A). Thus, an optimum collision-energy value which yields a remarkable difference in the qualifier ion ratio between leucine and isoleucine was present in any of the cases where the MRM transition was m/z 132.10>69.20, m/z 132.10>57.10, m/z 132.10>56.10, m/z 132.10>43.10, m/z 132.10>30.20 or m/z 132.10>27.20.

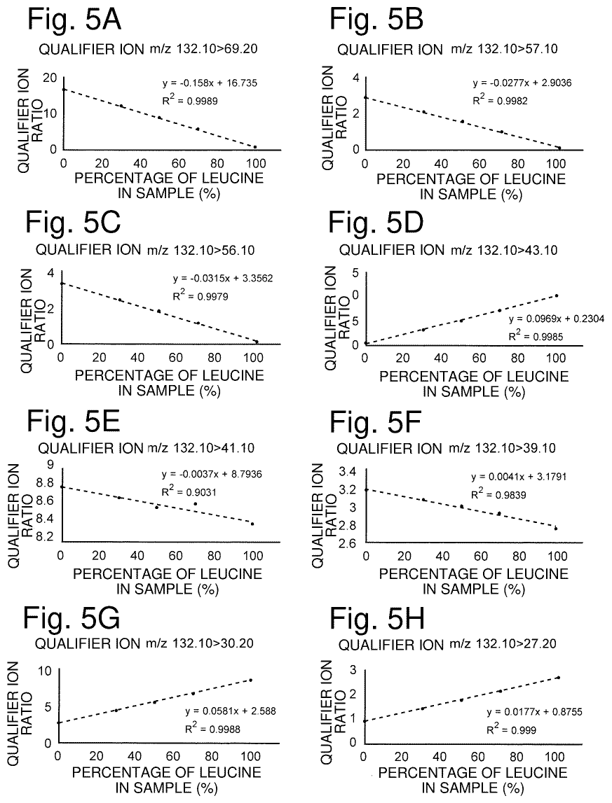

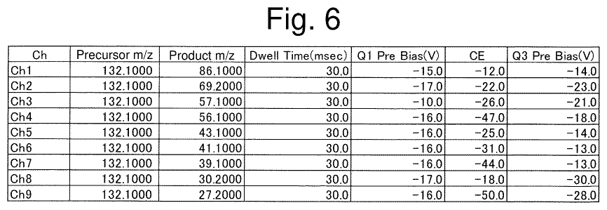

FIGS. 5A-5H are diagrams showing the relationship between the qualifier ion ratio and the percentage of leucine in leucine and isoleucine contained in a sample, as determined from the measured results, in the cases where the eight MRM transitions were used. FIG. 6 shows analysis conditions for the MRM measurements, in which the optimum value mentioned earlier is used as the collision energy in the case where the MRM transition is m/z 132.10>69.20, m/z 132.10>57.10, m/z 132.10>56.10, m/z 132.10>43.10, m/z 132.10>30.20 or m/z 132.10>27.20. As for m/z 132.10>41.10 and m/z 132.10>39.10, a collision-energy value is selected which yields a large difference in the qualifier ion ratio between leucine and isoleucine within a range where their qualifier ion ratios are at or close to the highest levels. As shown in FIGS. 5A-5H, the relationship between the percentage of leucine in leucine and isoleucine contained in the sample and the qualifier ion ratio could be expressed by a linear formula, i.e., a straight line. Accordingly, in the device according to the previously described embodiment, this relationship acquired beforehand by experiments is stored in the component determiner 33 as a calibration curve. When a qualifier ion ratio is acquired by an actual measurement of a sample, the component determiner 33 estimates the percentage of leucine or isoleucine by applying the acquired qualifier ion ratio to the calibration curve. Having a single relational expression obtained by using one of the eight MRM transitions and stored in the component determiner 33 as a calibration curve may be sufficient. It is also possible to have two or more relational expressions obtained by using two or more MRM transitions and stored in the component determiner 33 as calibration curves, in which case an average, median or similar value calculated from the estimated values respectively obtained by using those calibration curves can be used as the percentage of leucine or isoleucine.

### [Specific Example of Method for Discriminating Between Leucine and Isoleucine]

Initially, in the liquid chromatograph mass spectrometer according to the previously described embodiment, the measurement unit 2 under the control of the analysis control unit 4 repeatedly performs an MRM measurement for the MRM transition of a target ion and an MRM measurement for the MRM transition of a predetermined qualifier ion for the same unknown sample. The MRM measurements are repeatedly performed until a predetermined period of time is elapsed from the point in time of the injection of the sample by the injector 13. By this operation, a set of data forming a chromatogram waveform which represents the temporal change of the ion-intensity data is acquired as a measured result for each MRM transition and stored in the data collector 31.

The peak intensity calculator 32 creates a chromatogram waveform for each of the nine MRM transitions from the sets of data stored in the data collector 31, performs a peak detection process on each waveform and determines the peak-top value (peak intensity value). Although the peak intensity value is used in this example as the signal intensity for the calculating process (which will be described later), the peak area value from the beginning point to the ending point of the peak may be calculated for use as the signal intensity in place of the peak intensity value.

The component determiner 33 subsequently calculates the qualifier ion ratio, A2/A1×100, which is the ratio between the signal intensity A1 at the MRM transition corresponding to the target ion (m/z 132.10>86.10) and the signal intensity A2 at the MRM transition corresponding to the qualifier ion. Ultimately, the component determiner 33 applies this qualifier ion ratio to the calibration curve stored in the component determiner 33 to estimate the percentage of leucine or isoleucine.

### [Modes]

It is evident to a person skilled in the art that the previously described illustrative embodiments are specific examples of the following modes of the present invention.

(Clause 1) A method for analyzing leucine and isoleucine according to the present invention is a method for analyzing leucine and isoleucine using a tandem mass spectrometer, the method including:
a measurement step for acquiring intensity information of ions originating from leucine and isoleucine by performing, on a specimen, a first MRM measurement for at least one multiple reaction monitoring (MRM) transition selected from the group of m/z 132>69, m/z 132>57, m/z 132>56, m/z 132>43, m/z 132>41, m/z 132>39, m/z 132>30 and m/z 132>27 as well as a second MRM measurement for an MRM transition of m/z 132>86; and
a processing step for discriminating whether the specimen contains leucine or isoleucine, or for determining a relative abundance of leucine and isoleucine in the specimen, based on a qualifier ion ratio which is a ratio between a signal intensity obtained by the first MRM measurement and a signal intensity obtained by the second MRM measurement.

(Clause 9) A device for analyzing leucine and isoleucine according to the present invention includes:
a measurement unit which is a tandem mass spectrometer configured to perform, on a specimen, a first MRM measurement for at least one multiple reaction monitoring (MRM) transition selected from the group of m/z 132>69, m/z 132>57, m/z 132>56, m/z 132>43, m/z 132>41, m/z 132>39, m/z 132>30 and m/z 132>27 as well as a second MRM measurement for an MRM transition of m/z 132>86; and
a processing unit configured to discriminate whether the specimen contains leucine or isoleucine, or to determine a relative abundance of leucine and isoleucine in the specimen, based on a qualifier ion ratio which is a ratio between a signal intensity obtained by the first MRM measurement and a signal intensity obtained by the second MRM measurement in the measurement unit.

By the method for analyzing leucine and isoleucine according to Clause 1 and the device for analyzing leucine and isoleucine according to Clause 9, it is possible to discriminate whether the specimen contains leucine or isoleucine or to determine the relative abundance of leucine and isoleucine in the specimen based on the qualifier ion ratio which is the ratio between a signal intensity at the MRM transition of a target ion (m/z 132>86) which shows almost the same ion intensity for the same amount of target substance regardless of whether the target substance actually contained in the specimen is leucine or isoleucine and a signal intensity at the MRM transition of a qualifier ion (m/z 132>69, m/z 132>57, m/z 132>56, m/z 132>43, m/z 132>41, m/z 132>39, m/z 132>30 or m/z 132>27) which shows a different ion intensity for the same amount of target substance depending on whether the target substance actually contained in the specimen is leucine or isoleucine.

(Clause 2) In the method for analyzing leucine and isoleucine according to Clause 1,
the measurement step may include acquiring the intensity information of ions originating from leucine and isoleucine by performing the first MRM measurement and the second MRM measurement while varying collision energy, and
the processing step may include discriminating whether the specimen contains leucine or isoleucine based on the collision-energy dependency of the qualifier ion ratio.

By the method for analyzing leucine and isoleucine according to Clause 2, it is possible to discriminate between leucine and isoleucine even when there is no difference in the qualifier ion ratio between leucine and isoleucine at one value of collision energy.

(Clause 3) In the method for analyzing leucine and isoleucine according to Clause 1, the processing step may include estimating the relative abundance of leucine or isoleucine contained in a target specimen by using information representing a previously determined relationship between the qualifier ion ratio and the relative abundance of leucine or isoleucine in a specimen.

By the method for analyzing leucine and isoleucine according to Clause 3, it is possible to not only discriminate between leucine and isoleucine but also estimate the relative abundance of the two compounds.

(Clause 4) In the method for analyzing leucine and isoleucine according to one of Clauses 1-3, the transition for the first MRM measurement may be an MRM transition selected from the group of m/z 132>69, m/z 132>57, m/z 132>56, m/z 132>43, m/z 132>30 and m/z 132>27.

In the method for analyzing leucine and isoleucine according to Clause 4, there is a range of collision energy within which a significant difference occurs between the qualifier ion ratio for leucine and the qualifier ion ratio for isoleucine. By using the qualifier ion ratios obtained by performing the first and second MRM measurements while varying the collision energy within this range, the discrimination between leucine and isoleucine can be more assuredly achieved.

(Clause 5) In the method for analyzing leucine and isoleucine according to one of Clauses 1-3, the transition for the first MRM measurement may be m/z 132>41 or m/z 132>39.

In the method for analyzing leucine and isoleucine according to Clause 5, there is a range of collision energy within which the peaks of both leucine and isoleucine can be detected with a sufficient intensity while a clear difference is also observed between the qualifier ion ratio for leucine and the qualifier ion ratio for isoleucine. By using the qualifier ion ratios obtained by performing the first and second MRM measurements while varying the collision energy within this range, the discrimination between leucine and isoleucine can be assuredly achieved even when a foreign substance that possibly hinders the peak detection is contained in the specimen.

(Clause 6) In the method for analyzing leucine and isoleucine according to one of Clauses 1-5,
the measurement step may include repeatedly performing the first MRM measurement and the second MRM measurement, and
the processing step may use, as a measured result, a peak-top intensity value or a peak-area value of a peak showing a temporal change in ion intensity originating from leucine and isoleucine.

In the method for analyzing leucine and isoleucine according to Clause 6, when the height of the peak (peak-top intensity value) is used as the measured result, the processing can be quickly performed, so that a high level of screening efficiency can be achieved. On the other hand, the peak-area value normally yields a higher performance in quantitative determination than the height of the peak. Accordingly, when the peak-area value is used as the measured result, the discrimination or determination can be performed with a higher level of accuracy.

(Clause 7) In the method for analyzing leucine and isoleucine according to one of Clauses 1-6, a flow injection analysis method may be used for introducing a specimen into the tandem mass spectrometer.

The method for analyzing leucine and isoleucine according to Clause 7 does not require component separation by a column. This method reduces the measurement time per one specimen and is suitable for a screening test which requires quick processing.

(Clause 8) In the method for analyzing leucine and isoleucine according to one of Clauses 1-6, a liquid chromatograph may be used for separating components contained in the specimen before introducing the specimen into the tandem mass spectrometer.

The method for analyzing leucine and isoleucine according to Clause 8 additionally uses component separation by a column, so that an even higher level of accuracy in discrimination or determination can be achieved as compared to the case of using only the measured results obtained at specific MRM transitions.

### REFERENCE SIGNS LIST

1 . . . Liquid Chromatograph
11 . . . Mobile Phase Container
12 . . . Liquid Supply Pump
13 . . . Injector
2 . . . Mass Analyzer
201 . . . Ionization Chamber
202 . . . First Intermediate Vacuum Chamber
203 . . . Second Intermediate Vacuum Chamber
204 . . . High-Vacuum Chamber
21 . . . ESI Spray
22 . . . Desolvation Tube
23 . . . Ion Guide
24 . . . Skimmer
25 . . . Multipole Ion Guide
26 . . . Front Quadrupole Mass Filter
27 . . . Collision Cell
28 . . . Rear Quadrupole Mass Filter
29 . . . Ion Detector
3 . . . Data Processing Unit
31 . . . Data Collector
32 . . . Peak Intensity Calculator
33 . . . Component Determiner
4 . . . Analysis Control Unit
41 . . . Analysis Condition Storage Section
5 . . . Central Control Unit
6 . . . Input Unit
7 . . . Display Unit

1. A method for analyzing leucine and isoleucine using a tandem mass spectrometer, the method comprising:
a measurement step for acquiring intensity information of ions originating from leucine and isoleucine by performing, on a specimen, a first MRM measurement for at least one multiple reaction monitoring (MRM) transition selected from a group of m/z 132>69, m/z 132>57, m/z 132>56, m/z 132>43, m/z 132>41, m/z 132>39, m/z 132>30 and m/z 132>27 as well as a second MRM measurement for an MRM transition of m/z 132>86; and
a processing step for discriminating whether the specimen contains leucine or isoleucine, or for determining a relative abundance of leucine and isoleucine in the specimen, based on a qualifier ion ratio which is a ratio between a signal intensity obtained by the first MRM measurement and a signal intensity obtained by the second MRM measurement.

2. The method for analyzing leucine and isoleucine according to claim 1, wherein:
the measurement step includes acquiring the intensity information of ions originating from leucine and isoleucine by performing the first MRM measurement and the second MRM measurement while varying collision energy, and
the processing step includes discriminating whether the specimen contains leucine or isoleucine based on a collision-energy dependency of the qualifier ion ratio.

3. The method for analyzing leucine and isoleucine according to claim 1, wherein the processing step includes estimating the relative abundance of leucine or isoleucine contained in a target specimen by using information representing a previously determined relationship between the qualifier ion ratio and the relative abundance of leucine or isoleucine in a specimen.

4. The method for analyzing leucine and isoleucine according to claim 1, wherein the transition for the first MRM measurement is an MRM transition selected from a group of m/z 132>69, m/z 132>57, m/z 132>56, m/z 132>43, m/z 132>30 and m/z 132>27.

5. The method for analyzing leucine and isoleucine according to claim 1, wherein the transition for the first MRM measurement is m/z 132>41 or m/z 132>39.

6. The method for analyzing leucine and isoleucine according to claim 1, wherein:
the measurement step includes repeatedly performing the first MRM measurement and the second MRM measurement; and
the processing step uses, as a measured result, a peak-top intensity value or a peak-area value of a peak showing a temporal change in ion intensity originating from leucine and isoleucine.

7. The method for analyzing leucine and isoleucine according to claim 1, wherein a flow injection analysis method is used for introducing a specimen into the tandem mass spectrometer.

8. The method for analyzing leucine and isoleucine according to claim 1, wherein a liquid chromatograph is used for separating components contained in the specimen before introducing the specimen into the tandem mass spectrometer.

9. A device for analyzing leucine and isoleucine, comprising:
a measurement unit which is a tandem mass spectrometer configured to perform, on a specimen, a first MRM measurement for at least one multiple reaction monitoring (MRM) transition selected from the group of m/z 132>69, m/z 132>57, m/z 132>56, m/z 132>43, m/z 132>41, m/z 132>39, m/z 132>30 and m/z 132>27 as well as a second MRM measurement for an MRM transition of m/z 132>86; and
a processing unit configured to discriminate whether the specimen contains leucine or isoleucine, or to determine a relative abundance of leucine and isoleucine in the specimen, based on a qualifier ion ratio which is a ratio between a signal intensity obtained by the first MRM measurement and a signal intensity obtained by the second MRM measurement in the measurement unit.
